(19) United States
(12) Patent Application Publication
(10) Pub. No.: US 2016/0296739 A1
(43) Pub. Date: Oct. 13, 2016

(54) HORMONE OR OTHER PELLET DELIVERY DEVICE

(71) Applicant: Kenneth Edwards Cleveland, Jackson, MS (US)

(72) Inventor: Kenneth Edwards Cleveland, Jackson, MS (US)

(21) Appl. No.: 15/082,651

(22) Filed: Mar. 28, 2016

Related U.S. Application Data
(60) Provisional application No. 62/145,307, filed on Apr. 9, 2015.

(51) Int. Cl. A61M 37/00; A61M 5/28; A61M 5/315

(52) U.S. Cl. CPC A61M 37/0069; A61M 5/31526; A61M 5/28

(57) ABSTRACT

A pellet delivery device may be used to insert a plurality of pellets into a cavity or incision in a patient. The pellet delivery device includes a trocar and a plunger, and delivers pellets from an interior lumen of the trocar and through a distal opening along a sidewall of the trocar. The pellet delivery device includes a ratcheting system or other suitable mechanism to control pellet delivery, prevent plunger backout and/or provide tactile feedback during a pellet insertion process, enabling a user to insert one or more pellets at different locations within an incision of a patient without removing the trocar from the incision.

### CROSS-REFERENCE TO RELATED APPLICATION

This application claims the benefit of U.S. Provisional Application No. 62/145,307, filed Apr. 9, 2015, which is incorporated herein by reference in its entirety for all purposes.

### BACKGROUND OF THE INVENTION

In some procedures, slow-dissolving pellets of hormones such as testosterone or estrogen are inserted under the skin. The procedure involves numbing a region of the body, cutting a small incision, widening the incision with an insertion device, and pressing one or several pellets through the barrel of the device and then sealing the hole with surgical tape. In some cases, this process is repeated, involving the creation of several incisions to disperse the pellets around the desired area. Some methods of inserting multiple pellets into the same incision require removing the entire plunger from the insertion device, adding an additional pellet to the barrel, and reinserting the plunger into the barrel. The pellets dissolve over a period of months, releasing their payload.

### BRIEF SUMMARY OF THE INVENTION

Covered embodiments of the invention are defined by the claims, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

Disclosed herein are pellet delivery devices that may include a plunger and a trocar. The trocar may possess a distal end and a proximal end opposite an elongate, substantially cylindrical barrel having an interior lumen through which one or more pellets may be delivered. The interior lumen extends between the proximal end and a distal opening near a tip of the distal end. The trocar may also include a medial opening along the sidewall of the barrel that opens into the interior lumen, for example, to insert pellets into the lumen. The interior lumen accepts a plunger, which may have a flexible tip. The plunger and/or the interior lumen may also have a mechanism for controlling delivery of the pellets. For example, a mechanism of controlling delivery of the pellets may allow delivery of a subset of pellets at one time, such as single pellets, or groups of pellets. By way of another example, a mechanism of controlling delivery of the pellets may provide feedback (e.g. haptic feedback) to a user indicating when a pellet has been delivered. By way of a specific example, a mechanism of controlling delivery can include a ratchet mechanism including one or more ratchet teeth along a side of the plunger body. The ratchet teeth interact with a pawl connected with the trocar by a spring, so that the ratchet teeth can provide tactile or audible feedback to a user while the plunger is depressed through the lumen of the trocar.

Alternatively, or in addition, the pellet delivery device may include a pellet cartridge containing multiple pellets. The pellet cartridge includes an internal cavity for containing the pellets, a pellet cartridge opening arranged to communicate with the medial opening of the trocar, and a spring assembly for pressing pellets toward the opening. In some cases, the pellet cartridge is configured to deliver subsets of pellets or single pellets into the lumen of the trocar whenever the plunger is withdrawn past the medial opening.

Also disclosed are methods of using the pellet delivery device to deliver multiple pellets to different locations during the same insertion and within a single incision. The method includes inserting the device into an incision or other body cavity in a patient, and ejecting one or a subset of pellets into the incision or other cavity until the user receives the tactile or audible feedback from the device. The timing of the tactile or audible feedback relates to the ejection of one or a subset of pellets, signaling a user to displace or rotate the device so that the next pellet or subset of pellets may be ejected at a different site than the previous pellet or subset of pellets, but within the same incision or other cavity. The user may eject a series of pellets at predictable spacing within one incision or cavity without requiring additional incisions.

Alternatively, or in addition, a method of using the pellet delivery device with a pellet cartridge includes inserting the device into an incision or other body cavity in a patient, and ejecting a first pellet or first subset of pellets until the plunger is fully depressed. The plunger may be withdrawn, which permits the cartridge to press a second pellet or second subset of pellets into the lumen of the trocar. The plunger may be repeatedly depressed and withdrawn to insert multiple pellets or subsets of pellets. The delivery device may be rotated or translated between pellet insertions to deliver a series of pellets at predictable spacing within one incision or cavity without requiring additional incisions.

For a fuller understanding of the nature and advantages of the invention, reference should be made to the ensuing detailed description and accompanying drawings.

### BRIEF DESCRIPTION OF THE DRAWINGS

These and other features, embodiments, and advantages of the present disclosure are better understood when the following Detailed Description is read with reference to the accompanying drawings. The specification makes reference to the following appended figures, in which use of like reference numerals in different figures is intended to illustrate like or analogous components.

FIG. 1 is a side section view of a pellet delivery device having a trocar and a plunger, according to one example.

FIG. 2 is a side section view of the pellet delivery device of FIG. 1, with the plunger in a retracted position.

FIG. 3 is an end section view of the pellet delivery device of FIG. 1, showing the plunger in the barrel of the trocar.

FIG. 4 is a side section view of another example of a pellet delivery device, showing a long, flexible plunger tip bending at a distal opening of the trocar.

FIG. 5A-5F are schematic illustrations of a method of using a pellet delivery device to deliver a pellet subcutaneously in a patient according to some examples.

FIG. 5A shows the insertion of a pellet delivery device into an incision according to one example.

FIG. 5B shows the delivery of a first pellet to a first delivery site using the pellet delivery device of FIG. 5A.

FIG. 5C shows the rotation of the pellet delivery device of FIGS. 5A-5B to align the distal opening of the delivery device with a second delivery site, and the delivery of a second pellet to that second delivery site.

FIG. 5D shows the rotation of the delivery device and the delivery of a third pellet using the pellet delivery device of FIGS. 5A-5C.

FIG. 5E shows the rotation of the delivery device and the delivery of a fourth pellet using the pellet delivery device of FIGS. 5A-5D.

FIG. 5F shows the removal of the pellet delivery device of FIGS. 5A-5E from the incision.

FIG. 6 is a perspective view showing a pellet cartridge containing pellets for attaching with a pellet delivery device according to one example.

FIG. 7 is a side section view of another example of a pellet delivery device, showing a pellet cartridge connected with the trocar.

FIGS. 8A-8E are schematic illustrations of an exemplary method of using a pellet delivery device with a pellet cartridge to deliver a pellet subcutaneously in a patient.

FIG. 8A shows the insertion of a pellet delivery device with a pellet cartridge into an incision according to an example.

FIG. 8B shows the delivery of a first pellet into the incision using the pellet delivery device of FIG. 8A.

FIG. 8C shows the pellet delivery device of FIG. 8A loading a second pellet from the pellet cartridge into the trocar.

FIG. 8D shows the rotation of the delivery device of FIGS. 8A-8C.

FIG. 8E shows the delivery of a second pellet using the delivery device of FIGS. 8A-8D.

FIG. 9 shows a first example of a configuration of pellets in a patient.

FIG. 10 shows a second example of a configuration of pellets in a patient.

### DETAILED DESCRIPTION OF THE INVENTION

In the following description, various examples of the present invention will be described. For purposes of explanation, specific configurations and details are set forth to provide a thorough understanding of the embodiments. However, it will also be apparent to one skilled in the art that the invention may be practiced without the specific details. Furthermore, well-known features may be omitted or simplified so as not to obscure the example being described. The terms “invention,” “the invention,” “this invention” and “the present invention” used herein are intended to refer broadly to all of the subject matter of this patent application and the claims below. Statements containing these terms should be understood not to limit the subject matter described herein or to limit the meaning or scope of the patent claims below.

Described herein are pellet delivery devices designed to eject pellets from a side of a trocar instead of coaxially through the tip of the trocar. The tip of the barrel of the trocar is sharp, which may eliminate the need for a separate cannula. In use, a plunger, which may be flexible, forces a pellet around the curved trocar and out the side of the device. The user can then rotate the device and release another pellet. Rotating the device and ejecting a pellet out the side of the device (as opposed to coaxially through the tip of a barrel) allows a plurality of pellets to be inserted radially relative to a single incision, eliminating the need to create separate incisions for each pellet to be inserted. This reduces tissue damage and procedure time and provides more localized delivery of the contents of the pellets.

In some cases, the pellet delivery device includes a mechanism for controlling delivery of the pellets to influence the number of pellets that are delivered at one time. For example, the pellet delivery device may include a ratchet or other suitable mechanism for controlling advancement and ejection of the pellets or subset of pellets out of the device and into a user. If desired, the control mechanism may be designed to provide audible or tactile feedback to a user once a pellet or subset of pellets has been delivered.

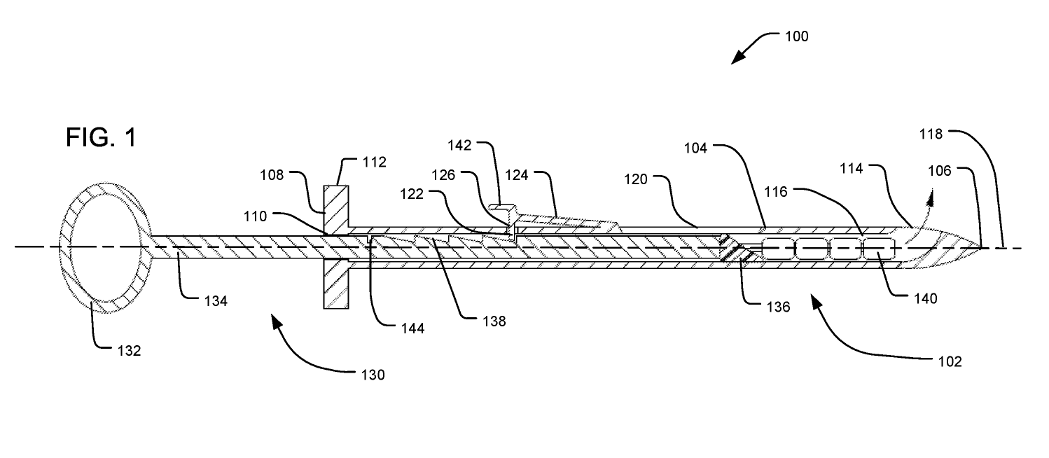

Referring now to the drawings, in which like reference numerals represent like parts throughout the several views, FIG. 1 shows a side section view of an example of a pellet delivery device 100. The pellet delivery device 100 has a trocar 102 and a plunger 130 removably fitted together. The pellet delivery device 100 is designed to deliver a series of pellets 140.

The trocar 102 includes a substantially hollow elongate barrel 104 having a sharp distal end 106 and an open proximal end 108 with a proximal opening 110 for accommodating the plunger 130. The distal end 106 is pointed for insertion into a bodily cavity, and the proximal end 108 has a flange 112 or other positive feature(s) that extends orthogonally from the elongate barrel 104 of the trocar 102 and facilitates grasping and positioning of the trocar 102. The trocar also includes an interior lumen 116 aligned with a longitudinal axis 118 and designed to receive a plunger 130, as discussed below. Near the tip of the distal end 106, a distal opening 114 connects the interior lumen 116 of the barrel 104 with the exterior of the trocar 102, exiting at an angle which may be up to 90 degrees to the longitudinal axis 118 of the trocar. Between the two ends 106, 108 of the barrel 104 of the trocar 102, a medial opening 120 also connects the interior lumen 116 to the exterior of the trocar 102. The pellets 140 may be loaded into the interior lumen 116 through the medial opening 120, or through the open proximal end 108. In the example illustrated in FIG. 1, a pawl 122 is connected with the trocar 102 by a spring 124, and the pawl extends into the interior lumen 116 of the barrel 104 of the trocar 102 through a small opening 126 in the barrel 104.

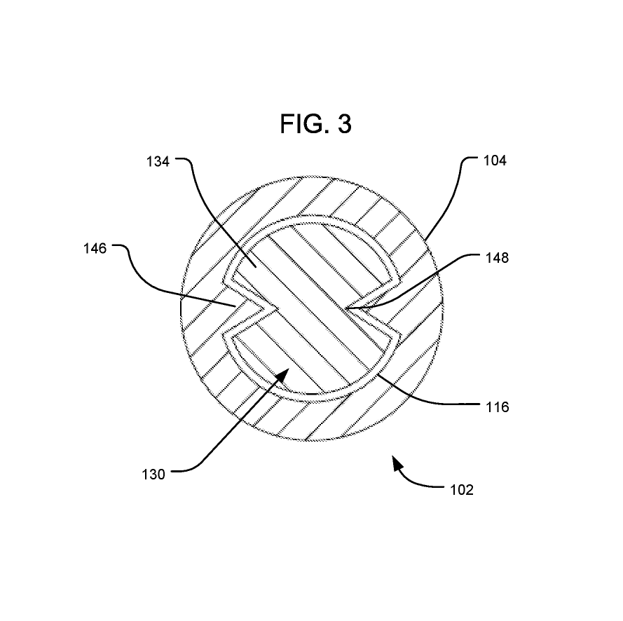

Plunger 130 is configured to be inserted into the interior lumen 116 and cooperate with one or more pellets 140 to be delivered through the trocar 102 and out of distal opening 114. Plunger 130 is an elongate and substantially cylindrical member having a handle end 132, a shaft 134, a tip 136, ratchet teeth 138, and surface features 148 (described below and illustrated in FIG. 3) for axially aligning the plunger 130 with the trocar 102. The length of the plunger shaft 134 is at least as long as the length of the interior lumen 116 of the trocar 102, so that when the plunger 130 is fully inserted into the lumen, the plunger tip 136 fully ejects pellets 140 from the distal opening 114 of the trocar. The plunger tip 136 may be flexible relative to the trocar 102, so that the tip can bend around a portion of the distal opening 114. The plunger tip 136 may also be scooped so that, when the plunger 130 is fully inserted into the interior lumen 116, the tip 136 may block at least a portion of the distal opening 114.

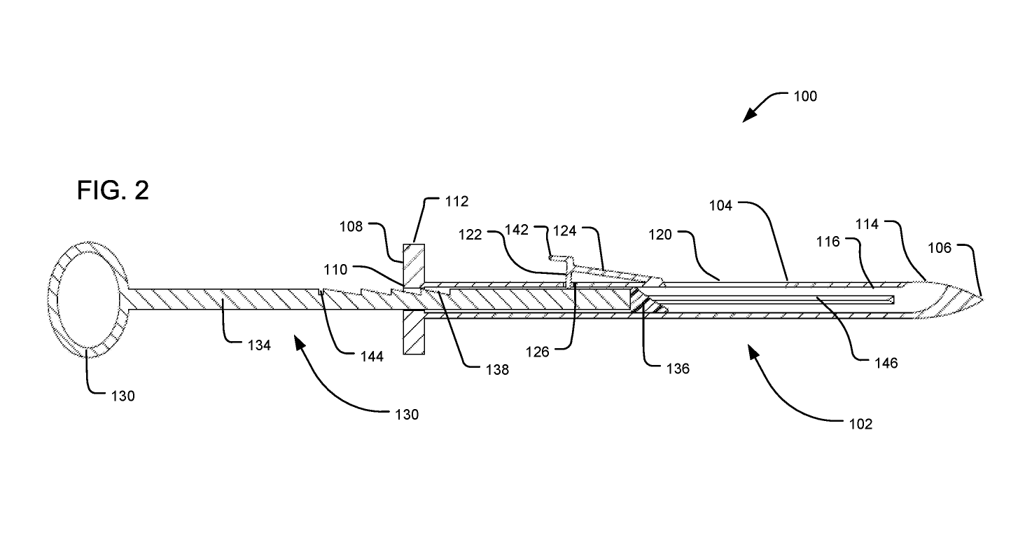

In some examples, the delivery device 100 includes a mechanism for controlling delivery of the pellets and for providing a user with feedback about such delivery. In the non-limiting example of FIG. 1, the control mechanism is in the form of ratchet teeth 138 positioned on the plunger shaft 134 such that the pawl 122 engages with the first of the ratchet teeth 138 when the plunger 130 is positioned behind a set of pellets in the elongate barrel 104. For example, in the pellet delivery device 100 shown in FIG. 1, ratchet teeth 138 are configured to begin engaging the pawl 122 when four pellets 140 of a predetermined size are positioned near the distal opening 114 and abut the tip 136 of the plunger 130. The lengths of each of the ratchet teeth 138 are substantially equal to the length of a pellet 140 (or the length of a predetermined number of pellets) so that the spring 124 will cause the pawl 122 to “click” down onto the next ratchet tooth after the ejection of each individual pellet (or predetermined number of pellets). As shown in FIGS. 1-2, the plunger shaft 134 may also have a groove 144 for catching the pawl 122 when the plunger 130 is fully inserted in the trocar 102 to prevent premature backout of the plunger when the device 100 is removed from, for example, an incision. Other variations of delivery control mechanisms and feedback mechanisms are possible.

In some examples, the geometry of the distal opening 114 partially obstructs the path of a pellet 140, so that loaded pellets are held in the lumen 116 of the barrel 104 of the trocar 102 until forcibly ejected. In other examples, the pellets 140 are held before ejection by a pliable surface feature (not shown) of the distal opening 114 or the lumen 116, by gravity (thus requiring that a user hold the loaded trocar 102 in an upright position with respect to the distal opening 114), or by any other suitable means or structure. In some cases, pellets 140 may be flexible, and may be sized relative to the distal opening 114 and lumen 116 such that the pellets must deform to pass out of the distal opening from the lumen.

The interior lumen 116 of the trocar 102 may be sized and shaped to accommodate any suitable medical pellet or implant. Pellets may be any size and type of suitable medical or veterinary pellet. For example, in conventional hormone replacement therapy, hormone pellets for men may be approximately 4.5 mm in diameter, and hormone pellets for women may be approximately 3.0 mm in diameter. Pellets for birth-control applications may be approximately 2.4 mm in diameter. Pellets for agricultural use, for example for delivering hormones to cattle, range in size from about 2 mm to about 5 mm. The disclosed devices may be configured with an interior lumen that accommodates any other suitable pellet size.

FIG. 2 shows the pellet delivery device 100 of FIG. 1 in a side section view with the plunger 130 in a retracted position. In this position, the plunger 130 is positioned so it does not obstruct medial opening 120 in the barrel 104 of the trocar 102, allowing for insertion of pellets 140 into the interior lumen 116 through medial 120. The pawl 122 is retracted from the ratchet teeth 138 manually by pulling upward at the spring 124 or using a pawl handle 142 attached to the spring 124. One or more positive surface feature(s) 146 in the interior lumen 116 are configured to interact with one or more negative surface feature(s) 148 (see FIG. 3) on the plunger shaft 134 of the plunger 130 for aligning the plunger in the trocar 102. Non-limiting examples of the positive and negative features include any suitable positive feature, e.g. a raised ridge, raised cylinder, raised bump, protrusion, etc.; and any suitable negative feature that receives the positive feature, e.g., a groove, slot, or comparable guide for receiving the positive feature. The pawl 122 or spring 124 may be attached with a levered ratchet release, so that a user may press on an end of the levered ratchet release to raise the pawl instead of lifting a handle.

FIG. 3 is an end section view of the pellet delivery device 100 of FIG. 1, showing the plunger 130 in the interior lumen 116 of the barrel 104 of the trocar 102. The interior lumen 116 includes positive surface features 146 in the interior lumen 116 for aligning the plunger 130. The plunger shaft 134 includes negative surface features 148 for receiving the positive surface features 146. The exemplary surface features 146 and 148 shown are a raised ridge 146 sized to slidingly fit within a groove 148. The ridge 146 prevents the plunger 130 from rotating within the barrel 104, but allows the plunger to slidingly displace in and out of the barrel. Although the surface features 146 as shown extend a length of the interior lumen 116, the surface features 146 may end at a region of the interior lumen 116 proximal to the handle end 132 from the medial opening 120, so that the pellets 140 may be inserted into the interior lumen 116 without being impeded by the surface features 146.

The plunger 130 may be aligned with the trocar barrel 103 using an alternative configuration of surface features within the interior lumen 116 of the barrel. For example, the negative surface features may be positioned along a length of the plunger 130 adjacent to the ratchet teeth 138, and the interior lumen 116 of the trocar barrel 103 may include the corresponding positive surface features. In some cases, the ratchet teeth may extend above a radius of the plunger 130 and into a void or cavity extending along a partial length of the trocar barrel 103.

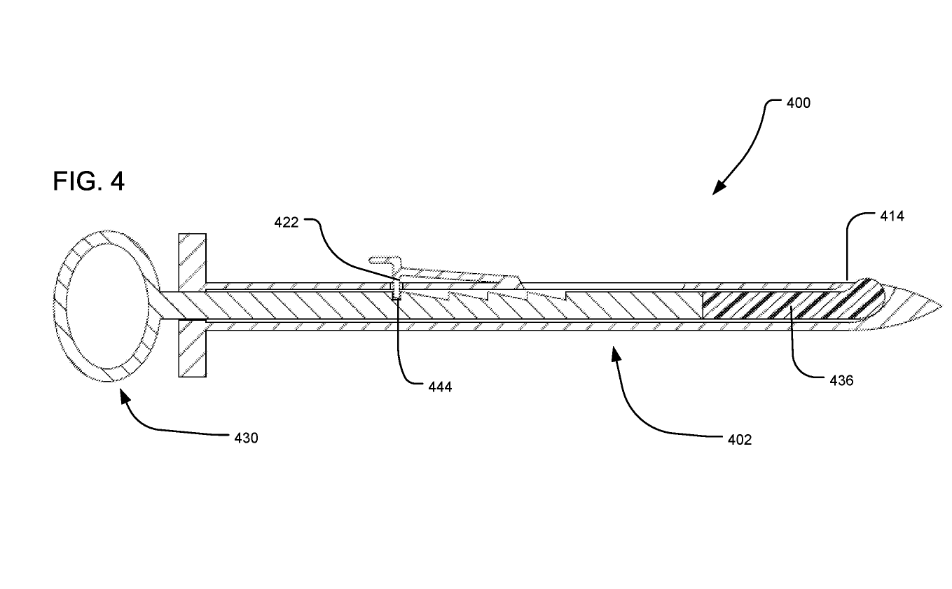

FIG. 4 is a side section view of another example of a pellet delivery device 400. The pellet delivery device 400 is similar to the device 100 of FIG. 1 in that it includes an elongate trocar 402 arranged to contain a plunger 430 with a ratcheting pawl 422. Delivery device 400 differs from the device 100 in that the plunger 430 terminates in a long and flexible plunger tip 436. The plunger tip 436 is configured to bend at a distal opening 414 of the trocar 402. As illustrated, the flexible plunger tip 436 obstructs the distal opening 414, preventing an ejected pellet from re-entering the distal opening 414 upon removal of the pellet delivery device 400 from a pellet delivery site in, for example, an incision.

Any suitable portion of the plunger 430 may be flexible. In some examples, as shown in FIG. 4, the plunger 430 may include a soft, flexible tip 436 configured to bend around a portion of the distal opening 414. In other examples, a longer portion of the plunger between the tip and the ratchet teeth may be flexible. In some cases, the entire plunger may be predominantly formed of a flexible material. In other cases, the plunger may be rigid, and the end may be shaped to partially obstruct a distal opening when the plunger is fully depressed. As shown in FIG. 4, the pawl 422 is held in a groove 444 of the plunger 430 for preventing backout of the plunger prior to removal of the pellet delivery device 400 from the pellet delivery site.

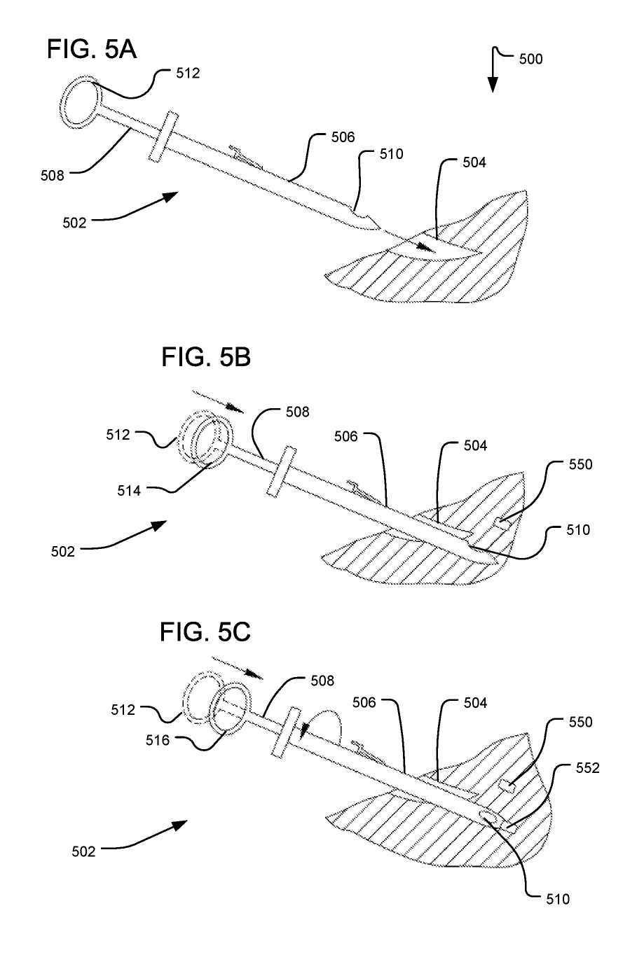

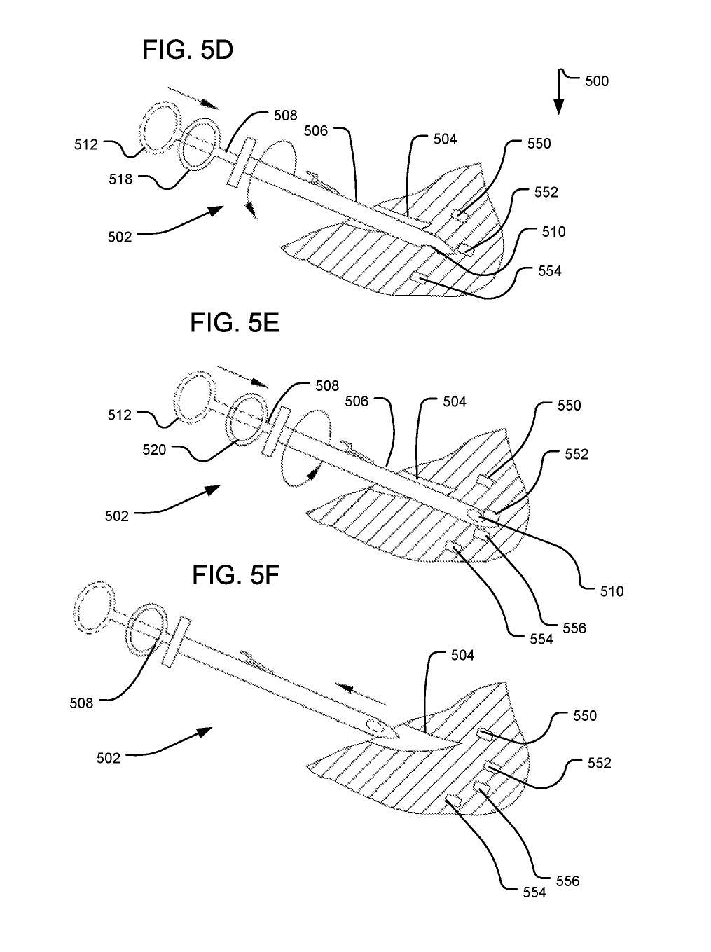

FIG. 5A-5F are schematic illustrations of an exemplary method 500 of using a pellet delivery device 502 to deliver one or more pellets subcutaneously in a patient.

FIG. 5A shows the insertion of a pellet delivery device 502 into an incision 504. As illustrated, pellet delivery device 502 includes a trocar 506, a plunger 508, and a distal opening 510. Insertion is preceded generally by the creation of a sterile field, local anesthesia (for example, by lidocaine), and the making of an incision. Four or any suitable number of pellets (not shown) are loaded in the pellet delivery device 502 as described above, and the plunger 508 is placed in a first position 512. Then the trocar 506 is inserted into the incision.

FIG. 5B shows the delivery of a first pellet 550 to a first delivery site using the pellet delivery device 502 of FIG. 5A. In this view, the trocar 506 is inserted into the incision 504, while the plunger 508 is in a first position 512 corresponding to the delivery device being fully loaded with pellets. The plunger 508 is pressed from the first position 512 to a second position 514, corresponding to a length of one of the pellets, ejecting a first pellet 550. In examples of the pellet delivery device 502 having a ratchet and pawl, the distance between the first and second positions 512 and 514 is also substantially equal to the length of a first ratchet tooth (see, e.g., ratchet teeth 138 in FIG. 1). As the plunger 508 is pressed into the trocar 506, one or more clicks may be felt or heard as the pawl passes over the ratchet teeth and as the first pellet 550 is deployed.

The degree of insertion of the pellet delivery device into a patient may vary. For example, the pellet delivery device 502 may be inserted up to any arbitrary depth according to the medical need. For example, the trocar 506 may be inserted for an entirety of an obturator portion of the trocar, which may be designated by markings or surface features on an exterior of the trocar, or the trocar may be partially inserted up to a predetermined depth.

FIG. 5C shows the rotation of the pellet delivery device 502 of FIGS. 5A-5B to align the distal opening 510 of the delivery device with a second delivery site within the incision, and the ejection of a second pellet 552. In this view, the pellet delivery device 502 is first rotated to a second orientation, which is a 90-degree rotation (or any desired degree of rotation) from the first orientation, and then the plunger 508 is pressed to a third position 516, corresponding to the length of two pellets from the original position 512. As above, in examples of a pellet delivery device having a ratchet and pawl, the distance between the second and third positions may also be substantially equal to the length of a second ratchet tooth (see, e.g., FIG. 1).

FIG. 5D shows the ejection of a third pellet 554 at a third delivery site within the incision using the pellet delivery device 502 of FIGS. 5A-5C. The pellet delivery device 502 is turned an additional 90 degrees (or other suitable rotation) to align the distal opening 510 with a third delivery site within the incision, and the plunger 508 is pressed from the third position to a fourth position 518, ejecting the third pellet 554. As above, in examples of a pellet delivery device having a ratchet and pawl, the distance between the second and third positions 516, 518 is also substantially equal to the length of a third ratchet tooth (see, e.g., FIG. 1).

FIG. 5E shows the ejection of a fourth pellet 556 at a fourth delivery site within the incision using the pellet delivery device 502 of FIGS. 5A-5D. As above, the pellet delivery device 502 is rotated an additional 90 degrees (or other suitable rotation) to align the distal opening 510 of the delivery device with a fourth delivery site within the incision, and the plunger 508 is depressed from a third position to a fourth position 520, ejecting the fourth pellet 556.

FIG. 5F shows the removal of the pellet delivery device 502 of FIGS. 5A-5E from the incision. In some cases, the plunger 508 includes an anti-backout surface feature, for example a locking groove at the handle end of the plunger relative to the ratchet teeth, so that the plunger is not pulled out of the trocar during the removal operation. Removal of a pellet delivery device is generally followed by the administration of pressure, icing, and sterile medical dressing.

The ratchet mechanism of the pellet delivery device 502 may have any suitable number of teeth, including a greater number of teeth than the number of pellets to be inserted. In this instance, the pellet delivery device 502 is loaded with a number of pellets, and the plunger 508 is pressed into the trocar 506 until a first pellet is visible at the distal opening 510. The method then proceeds as described above in operations 5A-5F.

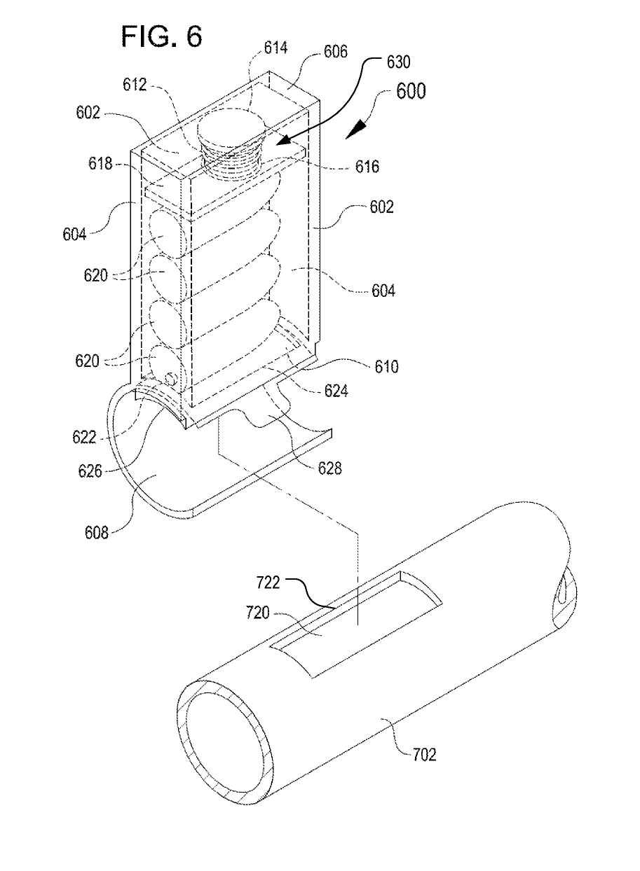

FIG. 6 illustrates a pellet cartridge 600 containing pellets 620 for attaching with a pellet delivery device trocar 702. The pellet cartridge 600 contains a plurality of pellets 620 and is configured to feed the pellets into the trocar 702 via a medial opening 720 sized to interact with the cartridge 600. The pellet cartridge 600 includes side walls 602 and end walls 604 spaced to form an interior cavity 610 that is sized to contain multiple pellets 620. In some cases, the spacing of the end walls and side walls 602, 604 is sufficient for the interior cavity 610 to contain a group of pellets 620 aligned in a single row, such that the pellets are consistently aligned with one another and with the trocar 702. In some cases, the side walls 604 may be spaced farther apart, such that the pellets 620 can align in an alternating, more closely packed formation.

The pellet cartridge 600 can connect with the trocar 702 via any suitable retention feature 608, such as for example, a flexible clip, strap, connector, flange, or other attachment means. As shown in FIG. 6, the retention feature 608 is configured to snap around the body of trocar 702 and connect the interior cavity 610 of the pellet cartridge 600 with the medial opening 720 of the trocar 702. The retention feature 608 may be elastically deformable, such that it urges the medial opening 720 into contact with the interior cavity 610 via the opening 624 of the cartridge 600. The pellet cartridge 600 may also include alignment feature(s) 626, which may interact with one or more receptive features (e.g., the perimeter 722 of the medial opening 720) of the trocar 702. As one non-limiting example, the alignment feature(s) 626 of the pellet cartridge 600 may be a projection of the cartridge that is sized to fit within the medial opening 720 of the trocar 702. The alignment feature(s) 626 of the pellet cartridge 600 may alternatively interact with other suitable features of the trocar 702, to align an opening 624 of the cartridge with the medial opening 720 of the trocar 702.

The pellet cartridge 600 further includes a dispensing assembly 630 for ejecting the pellets 620. The dispensing assembly 630 can be any suitable assembly for pushing pellets 620 out of the pellet cartridge 600. As shown in the non-limiting example of FIG. 6, the dispensing assembly 630 includes a spring-loaded assembly having a spring 612, a plate 618, and spring alignment features 614, 616 that connect or align the spring 612 with the plate 618 and with an interior of the top portion 606 of the pellet cartridge 600. The spring 612 is configured to press the plate 618 against the pellets 620 to force the pellets toward the opening 624 of the cartridge 600. A stopping feature 622, which may be a projection into the interior cavity 610 arranged to partially block the opening 622, may impede advancement of the pellets 620 out of the opening 624. In some cases, the stopping feature 622 is positioned to catch part of a pellet 620 such that one pellet is passed through the opening 624 at a time when the pellet cartridge 600 is in use. Other configurations to prevent advancement of a pellet may be used. For example, a film or cover 628 may be provided over the opening 624 and configured to prevent pellets 620 from exiting the cartridge 600 until the film is removed or pierced, and may further be configured to retain a sterile environment inside the cartridge interior 610. In some cases, the film or cover 628 is configured to be removed from the pellet cartridge 600 before use. In some cases, removal may occur after the pellet cartridge 600 is affixed to the trocar 702. In some cases, the pellet cartridge 600 is adapted to accommodate elongated, bullet-shaped pellets 620, which may prevent backout more easily than symmetrical pellets.

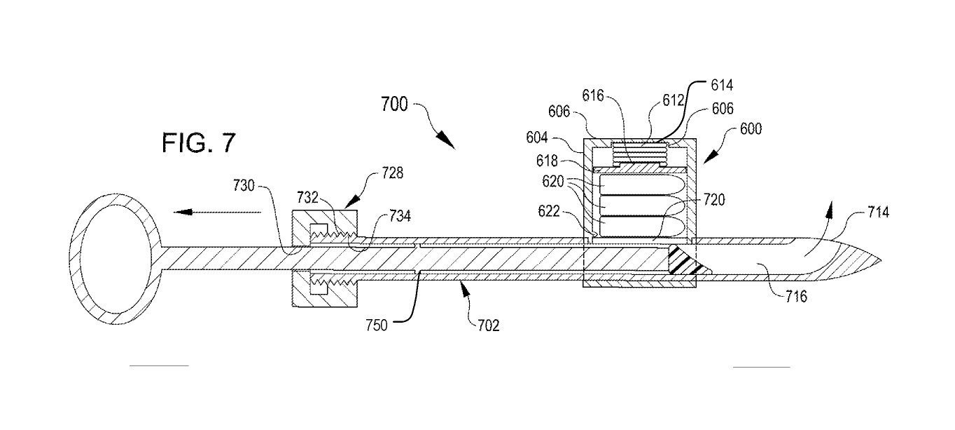

FIG. 7 illustrates a pellet delivery device 700 with the pellet cartridge 600 connected to the pellet delivery device trocar 702. The pellet delivery device 700 transfers pellets 620 from the pellet cartridge 600 into the trocar 702 through the medial opening 720. The medial opening 720 is sized and/or shaped to connect with the pellet cartridge 600. In some cases, the cartridge 600 is a removable part and/or a disposable part. For example, cartridge 600 may be pre-loaded with pellets 620 for use with the pellet delivery device 700, and may be replaced when emptied.

Similar to trocars 102 and 402, trocar 702 has an interior lumen 716 that serves as a pathway for the pellets 620 to exit the trocar through a distal opening 714 of the trocar 702. The interior lumen 716 has similar, or slightly larger, internal dimensions than the external dimensions of the pellets 620 such that the pellets can be readily pressed through the interior lumen 716. Similar to plungers 130 and 430, plunger 730 is an elongate and substantially cylindrical member with a plunger tip 736. Plunger 730 presses against the pellets 620 to advance them along the interior lumen 716 toward the distal opening 714. The plunger tip 736 can be shaped and/or flexible to better facilitate pressing a pellet 620 through the distal opening 714. In some cases, the plunger tip 736 has substantially the same diameter as an inner diameter of the interior lumen 716.

In some cases, the plunger 730 is connected with the trocar 702. As one example, the plunger 730 is connected with the trocar 702 by a cap 728 to prevent inadvertent backout of the plunger 730. The cap 728 can connect with the trocar 702 in any suitable way, such as via connecting features 734 of the cap and matching connecting features 732 of the trocar 702. In some cases, the connecting features 732, 734 may be threads, although the connecting features may include any suitable friction feature, interlocking mechanical part such as a clasp or snap, or other suitable connection or combinations thereof. In some cases, the plunger 730 includes an anti-backout feature 750 that interacts with the cap 728 to prevent backout. Anti-backout feature 750 can have any suitable configuration. As illustrated, anti-backout feature 750 is a protrusion from the plunger 730 that would be stopped by the cap 728.

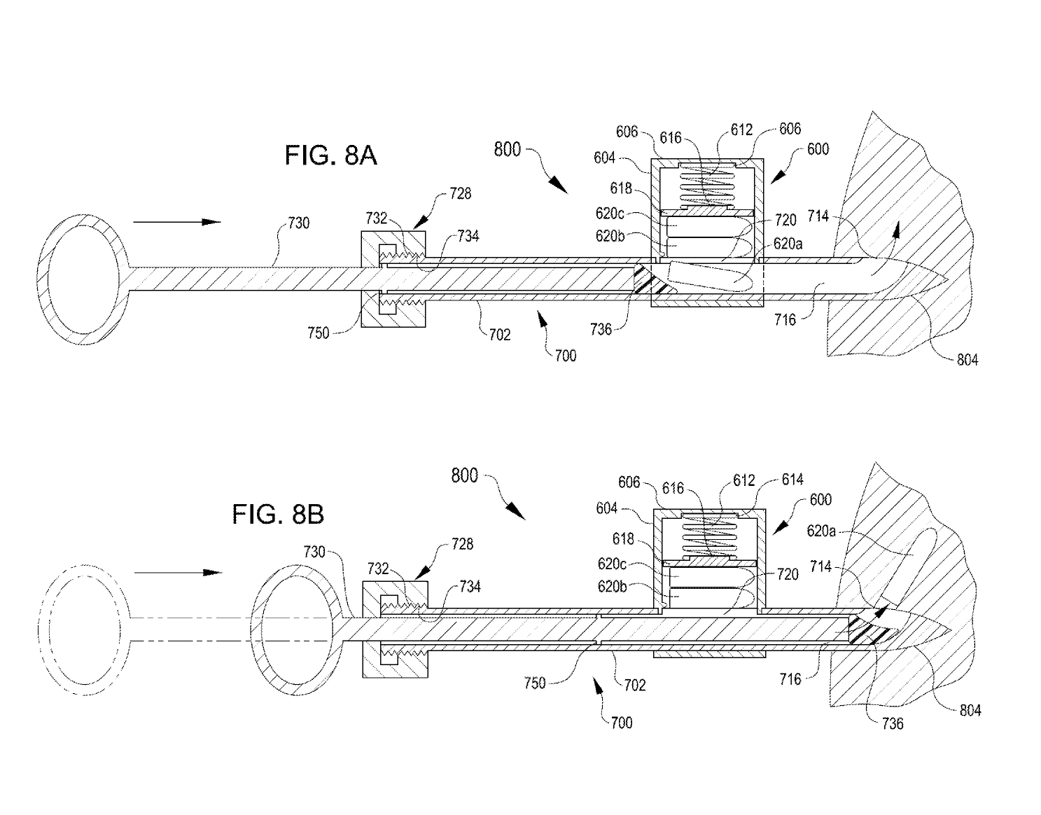

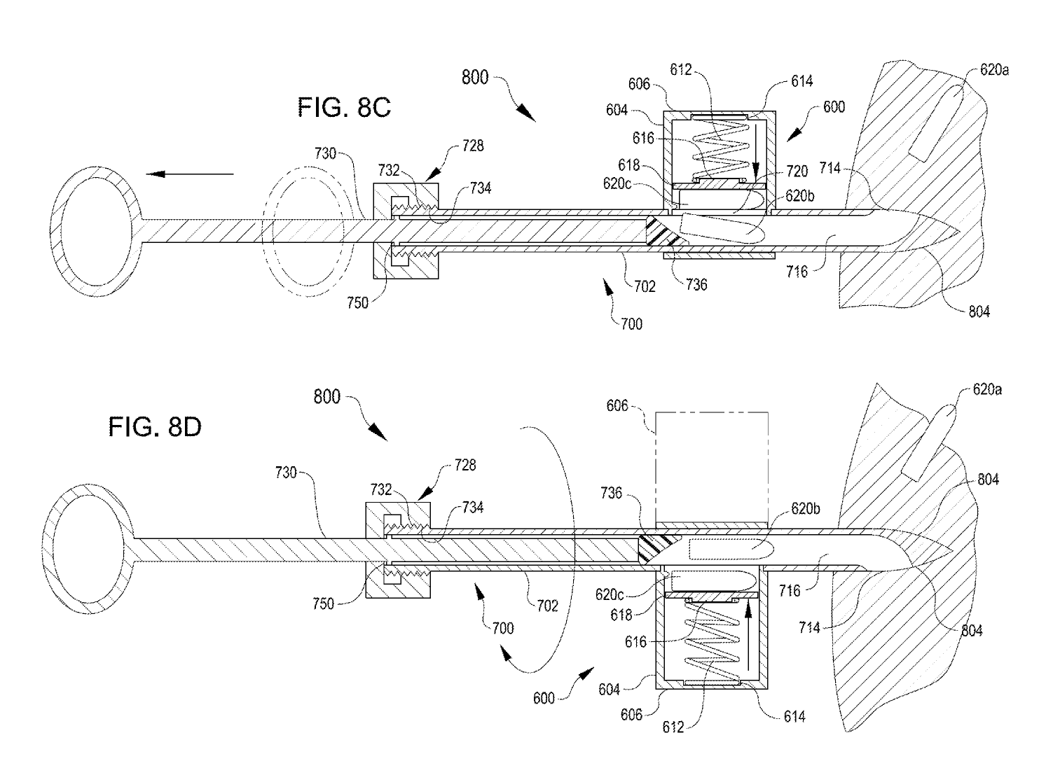

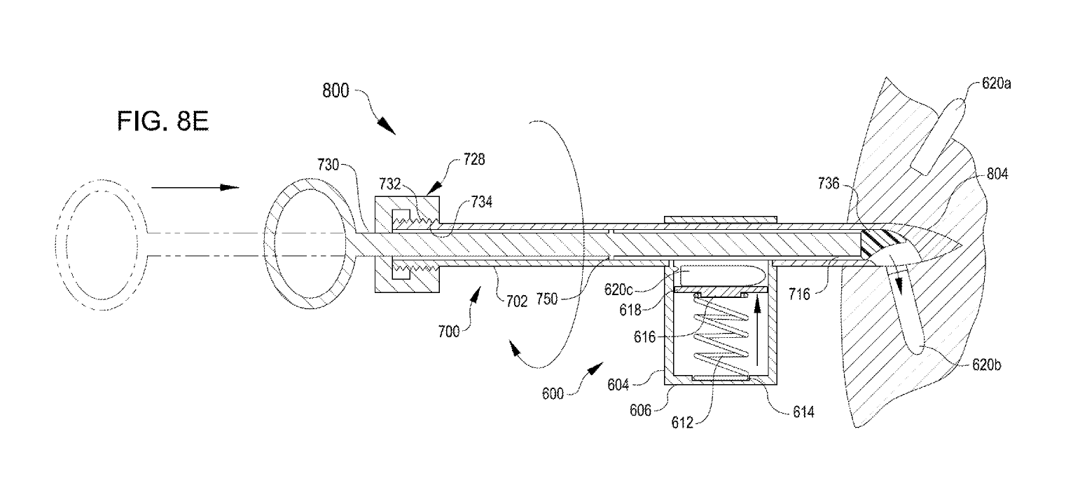

FIGS. 8A-8E are schematic illustrations of a method 800 of using the pellet delivery device 700 with the pellet cartridge 600 to deliver pellets 620 subcutaneously in a patient.

FIG. 8A shows the insertion of the pellet delivery device 700 with the pellet cartridge 600 into an incision. The view shows a simplified schematic of a pellet delivery device 700 with a trocar 702 penetrating patient tissue at an insertion 804. Insertion is performed generally as described above with reference to FIG. 5A. Any suitable number of pellets may be loaded into the pellet cartridge 600, e.g. 3 pellets as shown. In some cases, fewer or more than 3 pellets may be accommodated in the cartridge 600, depending on the specific dimensions of the pellets and cartridge.

The trocar 702 is inserted into the incision 804 such that the distal opening 714 is positioned within the incision for dispensing a pellet into the incision. A first pellet 620a is loaded into the lumen 716 of the delivery device 700 from the pellet cartridge 600. Specifically, while the plunger 730 is withdrawn past the medial opening 720, the spring 612 presses the pellets 620 toward the trocar 702, advancing the first pellet 620a into the interior lumen 716 of the trocar 702.

FIG. 8B shows the ejection of a first pellet 620a into the incision 804 using the pellet delivery device 700 of FIG. 8A. As shown, the plunger 730 is pressed into the trocar 702 such that the plunger tip 736 pushes the first pellet 620a out of the distal opening 714 and into tissue adjacent to the incision 804. The remaining pellets 620b-c remain in the cartridge 600.

FIG. 8C shows the pellet delivery device 700 of FIGS. 8A-B loading a second pellet 620b from the pellet cartridge 600 into the trocar 702. As the plunger 730 is withdrawn past the medial opening 720, the second pellet 620b is permitted to exit the pellet cartridge 600 into interior lumen 716. The spring 612 presses the third pellet 620c toward the trocar 702 as well, which presses the second pellet 620c in position relative the interior lumen 716.

FIG. 8D shows the rotation of the delivery device 700 of FIGS. 8A-8C to realign the distal opening 714 in a different direction within the incision 804. In some cases, multiple alignments may be used to accommodate multiple delivery sites from a single incision, as illustrated above with respect to FIGS. 5A-5E. The pellet cartridge 600 is configured to exert force on the pellets 620 by action of the spring 612. For example, the spring 612 can exert sufficient force that the delivery device 700 can be used in any orientation without pellets falling out of the device, or without impeding the loading of a pellet when the device is inverted.

FIG. 8E shows the ejection of a second pellet 620b using the delivery device of FIGS. 8A-8D. In this view, the plunger 730 is depressed to drive the second pellet 620b out of the distal opening 714 and into tissue at the incision 804. This and the above steps shown in FIGS. 8A-8D may be repeated for any suitable number of pellets according to a therapeutic need and/or according to a number of pellets stored in the pellet cartridge 600.

Any suitable number of pellets may be used, and the number of positions and ratchet teeth available on the plunger shaft may vary to suit other applications. Rotating the pellet delivery device about its axis may be repeated, alternating with the operation of pressing the plunger from one position to the next position, until all of a predetermined number of pellets within a pellet delivery device have been expelled into the incision.

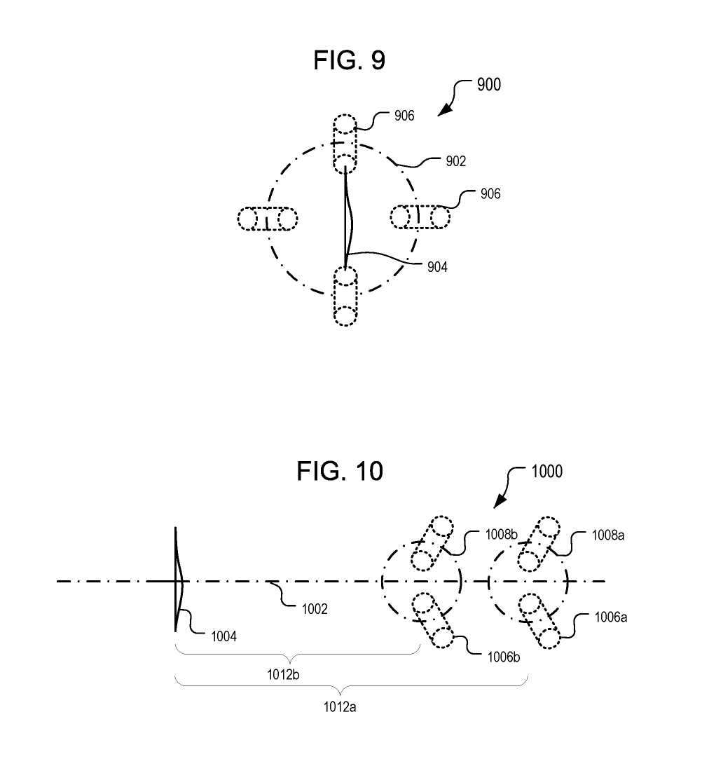

FIG. 9 shows an example of a first configuration 900 of pellets 906 in a patient. A delivery zone 902 is arranged around an incision 904 such that, when a delivery device (such as device 100 shown in FIG. 1) is inserted into the patient and deposits pellets 906, the pellets are arrayed within the delivery zone 902. The delivery zone 902 is shown as a circular region around the incision 904. Pellets 906 can be inserted into the delivery zone 902 via any suitable delivery method such as methods 500 and 800 (see FIGS. 5 and 8).

FIG. 10 shows an example of a second configuration 1000 of pellets 1006a, 1006b in a patient. A delivery device (such as device 100 shown in FIG. 1) can be inserted into a patient through an incision 1004 along a trocar path 1002. The trocar path 1002 can extend substantially parallel to and underneath the skin of a patient, or can be angled down into tissue of the patient, according to a medical need. Multiple delivery zones 1008a, 1008b may be located along the trocar path 1002. In one example, the delivery device is inserted at the incision site 1004 up to a first depth 1012a, and then releases a first payload of pellets 1006a (which may be one or more than one pellets) at one or more sites in the first delivery zone 1008a. The delivery device is then retracted to a second depth 1012b and releases a second payload of pellets 1006b (which may be one or more than pellets) within the second delivery zone 1008b, and so on.

In examples having a ratcheting mechanism, the device may have four or any suitable number of ratchet teeth and be configured to accept four or any suitable number of pellets having a length substantially similar to the length of the ratchet teeth, as discussed above. If the device possesses a ratchet, the device may have a number of ratchet teeth corresponding to the number of pellets to be dispensed and for pellets of any predetermined size. The trocar may be configured to accept a variety of plungers, and the plungers may be associated with packages of pellets of predetermined size and number.

In some cases, the length of each ratchet tooth may be substantially the same as the length of a predetermined number of pellets, so that a subset of pellets can be delivered at one time at each position within the incision. In some cases, the pellets may be sized to be delivered singly, or in multiples. For example, a subset of pellets may have a total length, when arranged end-to-end, approximately equal to a length of a single ratchet tooth. Accordingly, deliverable subsets of pellets can include single pellets, pairs of pellets, or any suitable number of pellets.

Any suitable alternative to a ratchet and pawl mechanism may be used to control the number of pellets that are delivered at one time and to provide feedback to a user regarding the delivery of pellets. As some non-limiting examples, any combination of markings, positive surface features, or both may be printed on or built into the plunger shaft. A user may be directed to press the plunger from a marked first position to one or more subsequent marked positions, the markings being visible from the exterior of the pellet delivery device.

As another non-limiting example, a rotating or screw-type mechanism may be used to control the delivery of pellets. In a rotating or screw-type mechanism, a trocar and a plunger may have features that interact to cause the plunger to translate into or out of the trocar when it is rotated relative to the trocar by a user. For example, a plunger may have a helical groove or a helical thread around a portion of the plunger shaft that interacts with a surface feature within the lumen of a trocar. By way of another example, a plunger may have a surface feature that interacts with a helical groove or a helical thread that extends into the lumen of the trocar from a sidewall of the trocar. Any such helical groove or helical thread may be arranged so as to associate a distance of translation of the plunger within the trocar with a predetermined number of rotations of the plunger within the trocar. For example, a helical groove or thread may be spaced such that one full rotation of the plunger in the trocar equates to a translation equivalent to a length of one pellet or subset of pellets. Thus, a user may be able to eject one pellet (or subset of pellets) at a time from the trocar by rotating the plunger by one full rotation within the trocar. Different numbers of rotations, or partial rotations, may be associated with a pellet length, or with a length of a set of pellets arranged end-to-end in the trocar barrel.

In some cases, a pellet delivery device includes a feeder tube capable of holding a plurality of pellets along with a ratchet or other device to control the pellet delivery so that a subset of the plurality of pellets can be delivered in a controlled manner in various locations within the tissue without needing to remove the device and reload it. In some cases, the ratchet may also act as a haptic device to provide audible or physically detectable feedback to the user. Once a pellet is delivered in the desired location, a ratchet or other suitable device engages and indicates to the user by an audible, visible, or physically detectable sensation that the pellet has been delivered. Thus, a user knows when to turn the device to prepare for delivery of the next pellet.

In some cases, the ratchet may be attached with a mechanism to prevent delivery of a subsequent pellet until the user rotates the trocar 90 degrees or by another predetermined amount. Once the device has been rotated by the predetermined amount, the ratchet mechanism permits delivery of the next pellet or subset of pellets at the new location. In this way, delivery of a subset of a plurality of pellets can be controlled and effected in more than one location in the body without needing to remove the device and reload it. This is in contrast to devices that are configured to accept and deliver only a single pellet at one time, or where a plurality of pellets can only be delivered at one time in a single location in the tissue, and thus reloading and repeated delivery attempts are required to deliver pellets in more than one location in the tissue.

In the following, further examples are described to facilitate the understanding of the invention:

### Example 1

There is provided a pellet delivery device, the device comprising a substantially hollow, elongate barrel comprising a pointed distal end, a proximal end, a sidewall between the proximal end and the distal end, and a lumen forming an interior of the barrel; a distal opening proximal to the distal end; a medial opening along a portion of the sidewall between the proximal end and the distal end; and a pawl coupled to a spring. The pawl extends into the barrel through the sidewall such that the pawl partially obstructs a portion of the lumen when the spring is relaxed. A plunger is provided, comprising a substantially elongate shaft having a cross-section sized to slidingly fit within the lumen, and a plurality of ratchet teeth arranged such that when the plunger is inserted in the trocar, the ratchet teeth engage with the pawl.

### Example 2

The component of any of the preceding or subsequent examples, wherein the distal opening is arranged along the sidewall of the barrel and opens substantially perpendicular to a longitudinal axis of the trocar and wherein a lumen wall curves toward the distal opening such that the lumen is in communication with the distal opening.

### Example 3

The component of any of the preceding or subsequent examples, further comprising a flange at the proximal end of the trocar.

### Example 4

The component of any of the preceding or subsequent examples, wherein: the lumen of the trocar further comprises a first alignment feature; and the plunger further comprises a second alignment feature, the first and second alignment features arranged to interact with each other and prevent rotation of the plunger with respect to the trocar when the plunger is inserted in the trocar.

### Example 5

The component of any of the preceding or subsequent examples, wherein a length of each of the plurality of ratchet teeth is approximately equal to a length of a pellet.

### Example 6

There is provided a pellet delivery device, the device comprising a trocar and a plunger. The trocar comprises a substantially hollow, elongate barrel comprising a pointed distal end, a proximal end, a sidewall between the proximal end and the distal end, and a lumen forming an interior of the barrel; a distal opening proximal to the distal end; and a medial opening at region of the sidewall between the proximal end and the distal end. The plunger comprises a substantially elongate shaft having a cross-section sized to slidingly fit within the lumen.

### Example 7

The component of any of the preceding or subsequent examples, wherein the medial opening is connectable with a pellet cartridge arranged to dispense one or more pellets into the lumen through the medial opening.

### Example 8

The component of any of the preceding or subsequent examples, wherein the distal opening is oriented substantially perpendicular to a longitudinal axis of the trocar; and the plunger further comprises a flexible tip, wherein the flexible tip is deformable when the plunger is inserted into the trocar such that the flexible tip bends toward the distal opening.

### Example 9

The component of any of the preceding or subsequent examples, wherein the flexible tip penetrates the distal opening when the plunger is fully inserted in the trocar.

### Example 10

The component of any of the preceding or subsequent examples, further comprising a pellet cartridge, the pellet cartridge comprising an internal cavity dimensioned to house one or more pellets; a cartridge opening arranged to communicate with the medial opening of the trocar; and a dispensing assembly that, when activated, urges the one or more pellets toward the medial opening of the trocar and into the lumen.

### Example 11

The component of any of the preceding or subsequent examples, wherein the pellet cartridge further comprises a retention feature arranged to wrap partially around the trocar and urge the cartridge opening against the medial opening.

### Example 12

The component of any of the preceding or subsequent examples, wherein the internal cavity of the pellet cartridge is arranged to contain the one or more pellets in a stack of pellets aligned with the cartridge opening.

### Example 13

The component of any of the preceding or subsequent examples, wherein the pellet cartridge further comprises a removable cover, the removable cover positioned to block the cartridge opening.

### Example 14

The component of any of the preceding or subsequent examples, wherein the trocar further comprises a connecting feature proximal to the proximal end of the barrel; and the plunger further comprises a cap slidingly connected to the plunger and connectable with the retention feature of the trocar.

### Example 15

The component of any of the preceding examples, wherein the plunger further comprises an anti-backout feature configured to interact with the cap for preventing backout of the plunger from the trocar.

### Example 16

There is provided a method of delivering a pellet using a pellet delivery device, the method comprising: receiving a first subset of one or more pellets into a lumen of a trocar of the pellet delivery device through a medial opening of the trocar, wherein the medial opening is located along a sidewall of the trocar between a proximal end and a distal end of the trocar; inserting the trocar into an incision of a patient so that a distal opening of the trocar aligns with a first delivery site wherein the distal opening is located along the sidewall of the trocar proximate the distal end; pressing a plunger into the lumen of the trocar until at least the first subset of the one or more pellets is ejected from the distal opening of the trocar into the first delivery site; rotating the delivery device in the incision of the patient so that the distal opening aligns with a second delivery site; and pressing the plunger into the trocar until at least a second subset of the one or more pellets is ejected from the distal opening of the trocar into the second delivery site within the incision.

### Example 17

The method of any of the preceding or subsequent examples, the method further comprising pressing the plunger into the trocar until a pawl of the barrel of the trocar passes from a first tooth of a plurality of ratchet teeth associated with the elongate shaft of the plunger to a second tooth of the plurality of ratchet teeth, wherein a length of the first tooth is similar to a length of the first subset of the one or more pellets being ejected from the distal opening into the first delivery site.

### Example 18

The method of any of the preceding or subsequent examples, the method further comprising pressing the plunger into the trocar until the pawl passes from a second tooth of the plurality of ratchet teeth to a third tooth of the plurality of ratchet teeth, wherein a length of the second tooth is similar to a length of the second subset of the one or more pellets being ejected from the distal opening of the trocar into the second delivery site

### Example 19

The method of any of the preceding or subsequent examples, the method further comprising: withdrawing the plunger so that the plunger substantially clears the medial opening of the trocar; and receiving the second subset of the one or more pellets into the lumen of the trocar via the medial opening from a pellet cartridge, wherein the pellet cartridge is configured to contain the one or more pellets in an internal cavity and urge the one or more pellets from the pellet cartridge and into the lumen by a dispensing assembly.

### Example 20

The method of any of the preceding examples, the method further comprising rotating the delivery device in the incision of the patient so that the distal opening aligns with a third delivery site; and pressing the plunger into the trocar until a third subset of the one or more pellets is ejected from the distal opening of the trocar into the third delivery site within the incision.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Various embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A pellet delivery device, comprising:
a trocar comprising:
a substantially hollow, elongate barrel comprising a pointed distal end, a proximal end, a sidewall between the proximal end and the distal end, and a lumen forming an interior of the barrel;
a distal opening proximal to the distal end;
a medial opening along a portion of the sidewall between the proximal end and the distal end; and
a pawl coupled to a spring, the pawl extending into the barrel through the sidewall such that the pawl partially obstructs a portion of the lumen when the spring is relaxed; and
a plunger comprising:
a substantially elongate shaft having a cross-section sized to slidingly fit within the lumen; and
a plurality of ratchet teeth arranged such that when the plunger is inserted in the trocar, the ratchet teeth engage with the pawl.

2. The pellet delivery device of claim 1, wherein the distal opening is arranged along the sidewall of the barrel and opens substantially perpendicular to a longitudinal axis of the trocar and wherein a lumen wall curves toward the distal opening such that the lumen is in communication with the distal opening.

3. The pellet delivery device of claim 1, further comprising:
a flange at the proximal end of the trocar.

4. The pellet delivery device of claim 1, wherein:
the lumen of the trocar further comprises a first alignment feature; and
the plunger further comprises a second alignment feature, the first and second alignment features arranged to interact with each other and prevent rotation of the plunger with respect to the trocar when the plunger is inserted in the trocar.

5. The pellet delivery device of claim 1, wherein a length of each of the plurality of ratchet teeth is approximately equal to a length of a pellet.

6. A pellet delivery device, comprising:
a trocar comprising:
a substantially hollow, elongate barrel comprising a pointed distal end, a proximal end, a sidewall between the proximal end and the distal end, and a lumen forming an interior of the barrel;
a distal opening proximal to the distal end; and
a medial opening at region of the sidewall between the proximal end and the distal end; and
a plunger comprising:
a substantially elongate shaft having a cross-section sized to slidingly fit within the lumen.

7. The pellet delivery device of claim 6, wherein the medial opening is connectable with a pellet cartridge arranged to dispense one or more pellets into the lumen through the medial opening.

8. The pellet delivery device of claim 6, wherein:
the distal opening is oriented substantially perpendicular to a longitudinal axis of the trocar; and
the plunger further comprises a flexible tip, wherein the flexible tip is deformable when the plunger is inserted into the trocar such that the flexible tip bends toward the distal opening.

9. The pellet delivery device of claim 8, wherein the flexible tip penetrates the distal opening when the plunger is fully inserted in the trocar.

10. The pellet delivery device of claim 6, further comprising:
a pellet cartridge comprising:
an internal cavity dimensioned to house one or more pellets;
a cartridge opening arranged to communicate with the medial opening of the trocar; and
a dispensing assembly that, when activated, urges the one or more pellets toward the medial opening of the trocar and into the lumen.

11. The pellet delivery device of claim 10, wherein the pellet cartridge further comprises:
a retention feature arranged to wrap partially around the trocar and urge the cartridge opening against the medial opening.

12. The pellet delivery device of claim 10, wherein the internal cavity of the pellet cartridge is arranged to contain the one or more pellets in a stack of pellets aligned with the cartridge opening.

13. The pellet delivery device of claim 10, wherein the pellet cartridge further comprises a removable cover, the removable cover positioned to block the cartridge opening.

14. The pellet delivery device of claim 6, wherein:
the trocar further comprises a connecting feature proximal to the proximal end of the barrel; and
the plunger further comprises a cap slidingly connected to the plunger and connectable with the retention feature of the trocar.

15. The pellet delivery device of claim 14, wherein the plunger further comprises an anti-backout feature configured to interact with the cap for preventing backout of the plunger from the trocar.

16. A method of delivering a pellet using a pellet delivery device, the method comprising:
receiving a first subset of one or more pellets into a lumen of a trocar of the pellet delivery device through a medial opening of the trocar, wherein the medial opening is located along a sidewall of the trocar between a proximal end and a distal end of the trocar;
inserting the trocar into an incision of a patient so that a distal opening of the trocar aligns with a first delivery site wherein the distal opening is located along the sidewall of the trocar proximate the distal end;
pressing a plunger into the lumen of the trocar until at least the first subset of the one or more pellets is ejected from the distal opening of the trocar into the first delivery site;
rotating the delivery device in the incision of the patient so that the distal opening aligns with a second delivery site; and
pressing the plunger into the trocar until at least a second subset of the one or more pellets is ejected from the distal opening of the trocar into the second delivery site within the incision.

17. The method of claim 16, the method further comprising:
pressing the plunger into the trocar until a pawl of the barrel of the trocar passes from a first tooth of a plurality of ratchet teeth associated with the elongate shaft of the plunger to a second tooth of the plurality of ratchet teeth, wherein a length of the first tooth is similar to a length of the first subset of the one or more pellets being ejected from the distal opening into the first delivery site.

18. The method of claim 16, further comprising:
pressing the plunger into the trocar until the pawl passes from a second tooth of the plurality of ratchet teeth to a third tooth of the plurality of ratchet teeth, wherein a length of the second tooth is similar to a length of the second subset of the one or more pellets being ejected from the distal opening of the trocar into the second delivery site.

19. The method of claim 16, further comprising:
withdrawing the plunger so that the plunger substantially clears the medial opening of the trocar; and
receiving the second subset of the one or more pellets into the lumen of the trocar via the medial opening from a pellet cartridge, wherein the pellet cartridge is configured to contain the one or more pellets in an internal cavity and urge the one or more pellets from the pellet cartridge and into the lumen by a dispensing assembly.

20. The method of claim 15, further comprising:
rotating the delivery device in the incision of the patient so that the distal opening aligns with a third delivery site; and
pressing the plunger into the trocar until a third subset of the one or more pellets is ejected from the distal opening of the trocar into the third delivery site within the incision.
